(19) United States
(12) Patent Application Publication
(10) Pub. No.: US 2024/0087548 A1
(43) Pub. Date: Mar. 14, 2024

(54) Tempo-Based Music Modulator

(71) Applicant: Stephane Guetin, Paris (FR)

(72) Inventor: Stephane Guetin, Paris (FR)

(21) Appl. No.: 17/931,075

(22) Filed: Sep. 9, 2022

(51) Int. Cl. G10H 1/00; A61M 21/02; G06F 3/16

(52) U.S. Cl. CPC G10H 1/0025; A61M 21/02; G06F 3/16; A61M 2021/0027; G10H 2210/111; G10H 2210/391; G10H 2220/371

(57) ABSTRACT

The present invention relates to a tempo-based music modulator configured to alleviate pain, anxiety, and other patient issues. Specifically, the present invention aims to modify the parameters of a music composition based on the intensity of the level of pain, anxiety, or fatigue and vital signs such as cardiac or respiratory frequency, blood pressure, or oxygen levels of the patient. The present invention provides a music modulator application that is configured to adjust to any level of pain, anxiety, pressure, heartbeat, blood pressure and generate music accordingly. A user can pre-select the level of condition based on the real-time condition experienced. The modulator will regulate its pre-programmed sound parameters based on the selection made by the user. The music produced by the modulator can modulate in relation to the different levels of user conditions computed by the algorithms, wherein each session is pre-configured based on the selection and condition of the user.

### CROSS-REFERENCE TO RELATED APPLICATION

This application claims benefit of U.S. Provisional Application No. 63/219,840, filed Jul. 9, 2021, which is incorporated by reference herein in its entirety.

### FIELD OF THE INVENTION

The present invention relates to a tempo-based music modulator configured to alleviate pain, anxiety, and other health-related issues. More specifically, the present invention aims to modify the parameters of a music composition based on the intensity of the level of pain, anxiety, or fatigue and vital signs such as cardiac or respiratory frequency, blood pressure, or oxygen levels of the patient as a method of treating the same.

### BACKGROUND

According to medical and health research, there is a correlating relationship between pain, anxiety, distress, and depression. Chronic pain is a significant health problem with significant emotional and social repercussions. Chronic pain is a leading cause of disability and one of the most common reasons for seeking medical care. More than a third of US adults with painful health conditions have indicated that pain moderately or severely interferes with activities of daily living.

Traditional pain treatment usually includes both physical and psychological treatment. Over the centuries, music and music therapy has been used as healing tool. Historically, the rise of an understanding of the body and mind in terms of the nervous system in the eighteenth century led to the emergence of a new wave of music for healing. Throughout the nineteenth century, an impressive number of books and articles were authored by physicians in Europe and the United States discussing the use of music as a therapeutic agent to treat both mental and physical illness. In the early 1990s, the re-introduction of music therapy and music medicine has been established in many medical procedures and particularly in the treatment of pain. It has been reported that music affects the cognitive component, diverting attention from pain, as emphasized by the Gate Control Theory. The gate control theory of pain asserts that non-painful input may close the nerve “gates” to painful input, preventing pain sensation from traveling to the central nervous system. A recent meta-analysis has highlighted the influence of music on cognitive functions, stating that music is the most effective mediator of emotional processing. This conclusion suggests that music that is pleasant for the patient may help to reduce the overall sensation of pain (Roy et al. 2008). Music therapy is thought to ease pain and anxiety primarily by distracting the patient and blocking pain pathways. Anxiety and pain are believed to be linked; thus, decreasing one may lead to decreases in the other. (Bradshaw, Brown, Cepeda, Pace 2011).

By its definition, pain is a subjective and highly personal experience, which presents challenges for both the researcher and clinician (Fillingim, 2017). Standardization of the technical, therapeutic music interventions to assist in managing pain can serve to be helpful as a treatment strategy. However, the main characteristic of a successful music medicine application seems to be related to the selection of a musical performance based on the patients' preference and the cultural background concerning the genre of music that is selected for therapy.

As a result, there is disclosure available on various music therapy methods and systems that claim to provide therapeutic relief. The relief is focused on alleviating people's health and well-being. The inventive techniques are applied to the challenging problem of reducing pain, anxiety, depression, and other health-related disorders as found in the prior art.

One such invention is disclosed in the US patent application publication US 2020/0243055 A1 to Muvik Labs LLC titled “Method and System for Musical Communication.” This prior art describes a system that generates music and audio associated with a state or an emotion within a communication for a user interface to generate and control music and audio characteristics associated with a state or a feeling. It discloses the communication which may be textual for identifying age, sex, or another state. The user selects the genres of music and the amount of time. The pain level of the user is then detected. The music is generated based on the current state and user selection by varying musical attributes. The musical attributes are changed to guide a user to a less painful state. It also discloses the database associated with a device. This disclosure does not, however, teach to storing recorded pain levels and using that recordation as a driving data parameter to adjust the music tempo and modulation to better treat the user's pain.

Another invention disclosed in US patent application publication US20070137463A1 to David Lumsden titled “Digital Music Composition Device, Composition Software and Method of Use.” This prior art describes personal portable groove generator device (MP3/PDA/Cellphone form), software, and website operation for truly novice users to create unique musically complete compositions, automatically or user-generated, through menu-directed selection from loop libraries to mix and build novel, complete, rich, musical grooves that can be audited, saved, uploaded/downloaded, evolved, and shared, P2P or via a community or other website. The prior art discloses the composition of music for music therapy and helps patients in pain and diseases. The user must first get authorized to access the account. The musical preference profiles and other profile types are requested from time to time. The invention mentions the music selection by the user and the acquisition of the patient's emotional, psychological, or cognitive state, wherein individually optimized music is provided according to the selection and state of the user. This disclosure does not, however, teach to storing recorded pain levels and using that recordation as a driving data parameter to adjust the music tempo and modulation to better treat the user's pain

Yet another invention is the Russian patent application publication RU2634672C2 to Zakrytoe, Aktsionernoe, Obshchestvo, Mediko-Biologicheskij, Nauchno-Issledovatelskij, and Tsentr Diskretnaya titled “Method for correction of CNS functional state in oncological patients by physical nature signals.” The prior art describes a method for correcting the functional state of the central nervous system in cancer patients with signals of a physical nature that include a series of sequential procedures. A preliminary recording of electroencephalographic signals of a biological object with hardware and then exposes it through headphones with test signals, which are a synthesized set of sounds. The prior art method allows to reduce the drug load and improve the functional state of the cancer patient, which is achieved by selecting a sound resonantly effective frequency range that can normalize the operating state of the central nervous system. This disclosure does not, however, teach to storing recorded pain levels and using that recordation as a driving data parameter to adjust the music tempo and modulation to better treat the user's pain.

Yet another invention is the US patent application publication US20200126524A1 to Life score Ltd titled “Apparatus and methods for cellular compositions.” This prior art provides systems, methods, and apparatus for cellular compositions/generating music in real-time using cells (i.e., short musical motifs). The cellular compositions are dependent on user data.

Although the 11-point numeric rating scale pain score is widely used to assess pain, this approach has limitations and improved tools are needed to guide the treatment of pain. Hence, there remains a greater need to modify the parameters of a music composition based on the intensity of the level of pain, anxiety, or fatigue and vital signs viz. cardiac or respiratory frequency, blood pressure, or oxygen levels of the patient. There is a need in the market to find a solution that can maximize the effect of the music intervention with tempo-based, unlimited sessions that treat various health ailments (pain, anxiety, or sleep disorder), and which can be solved by the present invention.

All the difficulties, disadvantages, and limitations of those mentioned earlier relevant and conventional prior arts can be overcome by the present invention, which has various technical advancements and economic benefits over the conventional prior art.

Distinct from the prior art, the present invention utilizes a patient directed digital treatment based on proprietary algorithms and machine learning (the “U Sequence”). The U Sequence of the present invention is a music therapy technique specifically developed to provide customized music solutions to the patient via the present invention software application. The ‘U Sequence’ scientifically modulates all the relevant musical parameters and uses the principles of hypno-analgesia. Hypnosis is likely to be effective for most people suffering from diverse forms of pain. It improves the overall management of pain and facilitates patient support. Furthermore, research shows that hypnosis works as part of a treatment program for several psychological and medical conditions, with pain relief being one of the most researched areas (Lynn et al., 2000). Among the benefits associated with hypnosis is altering the psychological components of the experience of pain that may cause even more severe pain. The U sequence musical protocols disclosed in the present invention will progressively bring the patient into a deep state of relaxation. It also naturally treats pain, anxiety, and sleep disorders.

Additionally, the present invention is not expensive to subscribe. It is easy to configure, effortless to maintain, and can be sold or subscribed to any patient with a reasonable cost, time, and operability.

### SUMMARY OF THE INVENTION

It is essential to the present disclosure, all embodiments are provided as illustrative and non-limiting representatives of various possible embodiments. In addition, the terms “is”, “can”, “will” and the like are herein uses as synonyms for an interchangeable with terms such as “may”, “may provide for”, and “it is contemplated that the present invention may” and so forth.

Furthermore, all elements listed by name such as music, tempo, modulation, pain, anxiety, fatigue, depression etc. encompass all equivalents for such elements. Such equivalents are contemplated for each element named herein.

For purposes of summarizing, certain aspects, advantages, and novel features of the present invention are provided herein. It is to be understood that not all aspects, advantages, or novel features may be provided in any one particular embodiment. Thus, the disclosed subject matter may be embodied or carried out in a manner that achieves or optimizes one aspect, advantages, or novel features or group of features without achieving all aspects, advantages, or novel features as may be taught or suggested.

In view of the foregoing disadvantages inherent in the known art, the present invention provides a novel solution to modify the parameters of a music composition based on the intensity of the level of pain, anxiety, or fatigue and vital signs such as cardiac or respiratory frequency, blood pressure, or oxygen levels of the patient. The purpose and intent of the invention are to maximize the effect of the music intervention session to treat the indication of pain, anxiety, or sleep disorder.

The features of the invention, which are believed to be novel, are particularly pointed out and distinctly claimed in the concluding portion of the specification. These and other features, aspects, and advantages of the present invention will become better understood with reference to the following drawings and detailed description.

The present invention provides a machine learning music modulator configured to adjust to any level of pain, anxiety, pressure, heartbeat, blood pressure and generate music accordingly. A user may pre-select the level of condition based on the real-time condition experienced.

In a preferred embodiment of the present invention, the machine learning music modulator may regulate the pre-programmed sound parameters based on the selection made by the user following the recordation and machine learning interpretation of how the user physiologically responds to sound and how that sound impacts the user's condition. The music produced by the modulator may modulate in relation to the different levels of user conditions computed by algorithms and adjusted by machine learning throughout the course of the treatment of the user. In an initial treatment session, a patient may experience pre-configured music modulations based on the data inputted by the patient at the start of the treatment session concerning the patient's symptoms, subjectively perceived pain level, or other relevant factors. During the course of the treatment session, biometric readers capture and assess a patient's pain level. In a preferred embodiment of the present invention, the biometric data is cataloged with the manual data inputted by the patient to establish a scale of pain and discomfort that, with subsequent sessions, allows the machine learning music modulator to adjust the modulated tempo to gradually reduce the level of pain of the patient.

As a means of operation, the front end of the machine learning music modulator catalogs the music content by genre organized by the backend of the machine learning music modulator as the endpoint output is read by the front end. After the patient selects the desired music content, the patient is prompted to enter the subjective pain level before starting the treatment which is graded on a scale of 0 to 10. The patient may then choose the duration of the song and initiate the session. Once the session is completed, the patient is prompted again to enter the subjective pain level on a scale of 0 to 10. During the treatment session, biometric readers also capture and assess the patient's pain level data. At the completion of the treatment session, the patient's manually inputted pain level data and biometric pain level data are recorded into the database.

In some embodiments, data input may include the desired music selection, the subjective pain levels, and any other data associated and inputted by the patient. Data output may principally be comprised of music files needed by the front end algorithm to play the music, the biometric pain assessment data captured by the biometric readers, and the machine learning algorithmic data that provides the music modulation protocols for following patient sessions. The backend also outputs one image file per music item. The session's pain level data is not displayed to the end-user. Only the application administrator can view the session data via the backend management tool.

In another preferred embodiment of the present invention, the following sound parameters may be modulated: Pitch (a low/high sound)=frequencies; Duration (a short/long sound)=tempo/rhythm; Volume, its intensity (a soft/strong sound); Timbre: everything that makes a sound produced at the same height, of the same duration, with the same intensity, does not resemble another.

In some embodiments, the modulator will regulate to its pre-programmed sound parameters based on the selection made by the patient. In other embodiments, the modulator will regulate to the algorithmically determined sound parameters based on the machine learning assessment of the biometric pain data and the patient's manually inputted subjective pain data. The music produced by the modulator can modulate in relation to the different levels of patient conditions computed by the algorithms, regardless of whether each session is pre-configured based on the selection and condition of the patient or if the session is configured based on the pain response patterns captured and analyzed by the machine learning assessment of the biometric pain data and the patient's manually inputted subjective pain data.

In some embodiments, if the manually inputted subject pain level entered by the patient is in the range between 3-6, the tempo of the music generated by the present invention may be 90. Conversely, if manually inputted subjective pain level entered by the patient is in the range between 6-10, the tempo of the music generated by the present invention may be 95.

In other embodiments, the present invention provides and Application Specification having a Front End Application which allows patient to listen to the music based on corresponding defined pain level; a custom-built Back Office showing all metrics for a given composition, account, or patient user and Phase 3 Module that allow medical representatives' networks to invite medical practitioners and doctors to join and prescribe the present invention to their patients.

The Front end Application of the Application Specification allows professionals and patients to listen to the present invention's compositions based on Music Genre on a mobile/tablet application or a web browser. Users are asked to authenticate using a prescriber code given to them by a medical professional. End users may be asked to enter a prescriber code to access the application for a monthly fee following a free trial. The patient user is prompted to identify the pain/anxiety, sleep aid, or awaking modes. Once the composition is selected, the patient user is prompted to enter the pain level before and after the session. The patient user may also leave comments on each track. Users may choose to modify the length of a session from 20 minutes to 60 minutes.

In the Back Office of the Application Specification the user and sessions data is managed by a custom-built back office showing all metrics for a given composition, account, or user. The Back Office provides three modules: 1. Back office management tool; 2. Professional Account Management tool and 3. Application Public Access & Training.

In one of the embodiment of the invention the Back Office of the Application Specification provides Back office management tool to implement the new features of the module. The credit card reporting system will be carried over the module's main functionality is to assist better and manage all relative content of music tracks. This includes mp3 files, artwork, instruments, descriptions, and comments. This module includes an extensive user management system that allows the present invention's administrator to create, edit, and delete multiple types of professional accounts. This management tool also consists of a statistical reporting of pain levels recorded per group, user, session, and track. This will include a search tool and dashboard for a clear overview of new accounts and the latest activity.

In another embodiment of the invention the Back Office of the Application Specification provides Professional Account management tool to allow professional institutions, medical practitioners, doctors and organizations to invite their patients or employees to join and use present invention software application. The present invention software application Back Office authenticates the accounts before they can be used. The tools allow its administrator to invite new patients by using a name and email address. The system also tells them who has accepted and who has not.

In an embodiment of the invention the Phase 3 Modules of the Application Specification provides Medical Representative Module. The present invention medical representative management tool allows medical representatives' networks to invite medical practitioners and doctors to join and prescribe the present invention application. The present invention application Back-Office will authenticate the medical representative accounts before use. The tools allow the general practitioners invited by the user to then invite new patients by using an email address—the tool reports to the medical representative which general practitioner or doctor accepted and who has not. The management tool will also include a statistical reporting of new accounts with patient counts. As well as pain levels before the session, after the session, average improvement, comments, and music listen per user, session, and track. The tool consists of reporting secured web pages, a search tool, and a dashboard for a clear overview of new accounts and the latest activity. An additional frequently asked questions section and or webpage will be added to the present invention marketing website to support and explain the different new features.

In an embodiment of the invention the Phase 3 Modules of the Application Specification provides General Practitioner and Doctors Module. The general practitioner and doctor management tool allows present invention administrators, group administrators (i.e., Sanofi), and medical representatives to invite new general practitioners and doctors to the present invention platform by using an email address. The invitation status will be shown in this same tool. Once the invitation is sent, The present invention application Back Office will send an invitation link via email. Once this link is clicked, the recipient is prompted to follow an E-learning video on a secured web page. The general practitioner or doctor can request support from present invention software application at any point in this process uses a prominent contact button. Once the e-learning is complete, users are asked to join present invention software application at no cost to access their management tool. The tool includes a dashboard and will report data on their patient's average pain levels before sessions, after sessions, music licensing patterns, and comments. The user will also receive access to a demonstration mode on the present invention software application to allow him or her to demonstrate the software application to a patient with a 10-30 second snippet of a song. The user has access to the update to a full license at a standard subscription cost. An additional frequently asked questions section and or webpage will be added to the present invention marketing website to support and explain the different new features.

In an embodiment of the invention the Phase 3 Modules of the Application Specification provides Patient Health Record Refactor. The present invention application Back Office is refactored to accept static and dynamic patient health questions. The static questions will be asked at the signup stage for a patient. The patient user will no need to update these records. The dynamic questions are asked to the patient periodically using a notification sent to the application installed on their phone. These questions can be modified or added at any time in the present invention application Back Office by a present invention administrator. The new health records and the Music Listening application include a new statistics section showing critical data to the user, such as how many sessions played, average pain before, average pain after, most listened to songs, and average improvements.

In an embodiment of the invention the Phase 3 Modules of the Application Specification provides Group Admin. The present invention application Back Office is refactored to allow a group administration user to view the funnels they created with medical representatives, general practitioners, doctors, and their patients. This tool reports back the pain levels before, pain levels after, average improvements, and music listen within each funnel broken down using a tree system. The dashboard gives a global overview of how its network is performing. Once in the reporting sections, the group admin can review a specific funnel and its performance. The group's funnel data can be exported to a CSV file at the group admin representative's request.

Another preferred embodiment the instant invention provides Technical Specification having: 1. Development Stack; and 2.Documentation & code backups.

In preferred embodiment of the invention the Technical Specification provides Development Stack. The present invention application development environment and web server are deployed and runs on Amazon AWS and Google Cloud. Also, any and all database migrations from the old current system to the newly deployed system built by Cerebral Digital Inc. are performed using the latest best practices with the highest encryption available. The following is the breakdown of the stack. API endpoint smoke tests and security vulnerability testing tools are used to ensure the stability of all systems with redundancy. Frontend: ReactJS, Cordova. Backend: NodeJS, MongoDB database, Amazon Cognito. 3. Testing: Assertible.com, Netsparker.com.

In preferred embodiment of the invention the Technical Specification provides Documentation & code backups. The source code is documented in Github repositories based on the front and back end. The repositories will include “read me” files with deployment instructions, so external developers are up to date on building and deploying the applications available locally and on servers. System and database backups are managed using Amazon Web Services.

The embodiments of the invention described herein are exemplary and numerous modifications, variations, and rearrangements can be readily envisioned to achieve substantially equivalent results, all of which are intended to be embraced within the spirit and scope of the invention. Furthermore, while the preferred embodiment of the invention has been described in terms of the components and configurations, it is understood that the invention is not limited to those specific dimensions or configurations but is to be accorded the full breadth of the spirit of the invention.

### BRIEF DESCRIPTION OF THE DRAWINGS

These and other features, aspects and advantages of the present invention will become better understood with regard to the following description, appended claims, and accompanying figures.

FIG. 1 is a schematic that illustrates the system process architecture of the present invention.

### DETAILED DESCRIPTION

The present invention overcomes the limitations of the prior art by providing a novel solution to modify the parameters of a music composition based on the intensity of the level of pain, anxiety, or fatigue and vital signs such as cardiac or respiratory frequency, blood pressure, or oxygen levels of the patient.

It is essential to understand that the drawings and the associated descriptions are provided to illustrate potential embodiments of the invention and not to limit the scope of the invention. Reference in the specification to “one embodiment” or “an embodiment” is intended to indicate that a particular feature, structure, or characteristics described in connection with the embodiment is included in at least an embodiment of the invention. The appearances of the phrases “in one embodiment” or “an embodiment” in various places in the specification are not necessarily all referring to the same embodiment.

As used in this disclosure, except where the context requires otherwise, the term “comprise” and various of the term, such as “comprising”, “comprises” and “comprised” are not intended to exclude other additives, components, integers or steps.

In the following description, specific details are given to provide a thorough understanding of the embodiments. However, it will be understood by one of ordinary skill in the art that the embodiments may be practiced without these specific details. Well-known features, elements or techniques may be shown in detail in order not to obscure the embodiments.

The system of the present invention is suitable for modifying the parameters of a music composition based on the intensity of the level of pain, anxiety, or fatigue and vital signs such as cardiac or respiratory frequency, blood pressure, or oxygen levels of the patient

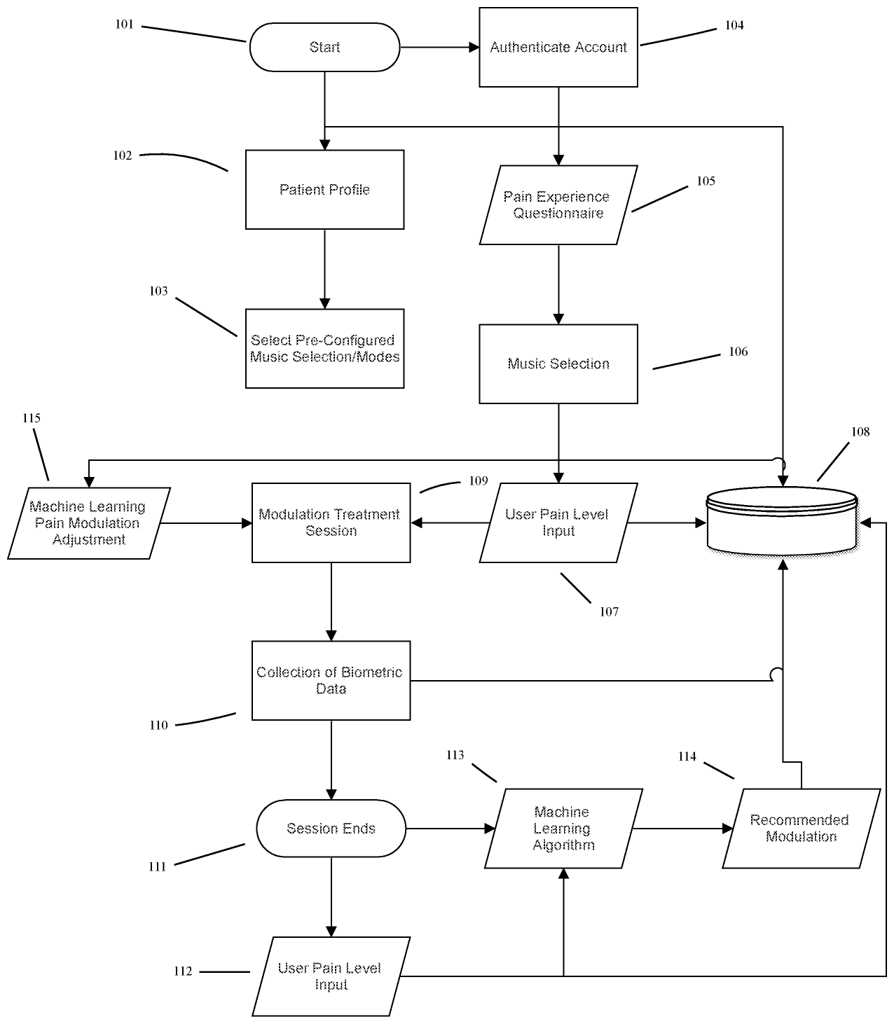

FIG. 1 shows a schematic that illustrates the system process architecture of the present invention. A viewer may perceive that the tempo-based music modulator allows a user to 101 initiate the login process by creating a 102 patient profile and 103 selecting a preferred and musical selection upon the creation of the patent user's profile. The 102 patient profile information is stored in the 108 database and may be shared with a medical professional who may provide additional medical input to the 102 patient's profile. Once created, a patient user may 101 initiate the login process and 104 authenticate their account. Once 104 authenticated, a patient user may be prompted to complete a 105 questionnaire regarding their pain experience. This 105 can be completed at the start of a patient user's first 109 modulation treatment session or in a series of 109 modulation treatment sessions. A patient user may also elect to 106 modify or confirm the patient user's previously selected 103 music selection and profile modes.

Prior to the start of the 109 modulation treatment session, the patient user is prompted to 107 input the patient user's current subjective pain level input. The 107 patient user's pain level drives the preferred music tempo and modulation. By way of non-limiting example, if the manually inputted subject pain level entered by the patient user is in the range between 3-6, the tempo of the music generated by the present invention may be 90. Conversely, if manually inputted subjective pain level entered by the patient user is in the range between 6-10, the tempo of the music generated by the present invention may be 95.

In a preferred embodiment of the present invention, the patient user is monitored using biometric readers to collect 110 biometric data to assess a patient user's pain level during the length of the 109 modulation treatment session. The 110 collected biometric data is stored in the 108 database which is made part of the patient user's 102 patient profile.

At the termination of the 109 modulation treatment session, a patient user may be prompted to 112 input the patient user's then subjective pain level at the conclusion of the 109 modulation treatment session. A 113 machine learning algorithm analyzes the 112 pain level collected at the end of the session and the 110 biometric data that is collected during the course of the 109 modulation treatment session to provide a 114 recommended modulated music tempo that, based on the 102 patient user's profile, the patient user's 105 pain experience questionnaire, the 107 pain level input at the start of the 109 modulation treatment session, the 112 pain level input at the end of the 109 modulation treatment session, and the 110 biometric data can 115 adjust the music temp during a 109 modulation treatment session in order to assist the patient user to receive treatment that is uniquely tailored to the patient user's pain level, treatment goals, and medical recommendations. By way of non-limiting example, a medical professional may receive progress reports on the patient user's 109 modulation treatment sessions and may input data (not shown) into the 102 patient user's profile that is stored in the 108 database. This medical professional input may be used by the machine learning algorithm to adjust the music temp during the 109 modulation treatment session.

The present invention, when used over the course of a series of 109 modulation treatment sessions, may result in the treatment, alleviation, and/or reduction of pain, anxiety, and other health-related issues.

Although the present invention has been described with a degree of particularity, it is understood that the present disclosure has been made by way of example and that other versions are possible. As various changes could be made in the above description without departing from the scope of the invention, it is intended that all matter contained in the above description or shown in the accompanying drawings shall be illustrative and not used in a limiting sense. The spirit and scope of the appended claims should not be limited to the description of the preferred versions contained in the disclosure.

All features disclosed in the specification, including the claims, abstracts, and drawings, and all steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent or some similar purpose, unless expressly stated otherwise. Thus, unless expressly stated otherwise, each feature disclosed is one example only of a generic series of equivalent or similar features.

While the present invention generally described herein has been disclosed in connection with a number of embodiments shown and described in detail, various modifications should be readily apparent to those of skill in the art.

What is claimed is:

1. A tempo-based music modulator system and application comprising:
an application specification having a front-end application, a custom-built back office, and a phase 3 module that allows a patient user to listen to music at a tempo based on the pain level of the patient user;
a technical specification comprising a development stack, documentation, and code backups;
a user profile;
user pain input;
a database;
a biometric reader;
a modulator that is configured to modulate the sound parameters of the music according to the pain level of the patient user whereby such sound parameters include pitch, frequency, duration, tempo/rhythm, volume, intensity, and the timbre of the music generated;
a non-transitory computer-readable medium storing program; and
a machine learning algorithm.

2. The tempo-based music modulator application of claim 1 wherein the music modulator modifies the parameters of a music composition based on the intensity of the level of pain, anxiety, or fatigue and vital signs of the user such as cardiac or respiratory frequency, blood pressure, or oxygen levels.

3. The tempo-based music modulator application of claim 1 wherein the front end catalogs the music content by genre organized by the backend in the endpoint output being read by the front end, the user selects music and enters the user's pain before starting on a scale of 0 to 10 further the user chooses the duration of the song and start the session, and on completion of the session, the user is prompted again to enter their pain level on a scale of 0 to 10.

4. The tempo-based music modulator of claim 1 wherein user's profile, music selection, pain level prior to a modulation session, pain level at the conclusion of a modulation session, biometric data, and length of a session are recorded on the database and stored as a session.

5. The tempo-based music modulator of claim 1 where the machine learning algorithm analyzes and modifies music tempos within the modulator.

6. The tempo based music modulator of claim 5 wherein the machine learning algorithm comprises of one or more processors and a data storage coupled to one or more processors, having instructions stored thereon which, when executed by at least one processor, causes the one or more processors to perform operations comprising: receiving analytic pain level data; receiving biometric user data; identifying the analytic difference between at least two pain level data points; generating analytic differences between at the least two pain level data points and the biometric user data; and generating a modification to the pre-configured music tempo established by the user's manual pain level data entries.

7. The system of claim 5, whereby the analytical difference between the at least two pain level data points and that analytical difference between the at least two pain level data points and the biometric user data may be received as at least one sequence of data, wherein said sequence of data may be maintained in a historical database.

8. The system of claim 7, wherein the system is configured to classify the at least one sequence of data and the system is respectively trained to use the at least one sequence of data as a training data set.

9. The system of claim 7, whereby the system is configured to compute the divergence between the numerical representation and a second set of at least one other sequence of data, wherein the second set of at least one other sequence of data comprises the modulation modification.

10. The system of claim 9, whereby the system analyzes the computed divergence between the numeric representation and the second set of at least one other sequence of data, and generates a music tempo modulation based on the computed divergence.
